(19) United States
(12) Patent Application Publication
(10) Pub. No.: US 2008/0033524 A1
(43) Pub. Date: Feb. 7, 2008

(54) Heated balloon assembly for delivery of polymeric stents

(76) Inventor: David C. Gale, San Jose, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/827,341

(22) Filed: Jul. 10, 2007

Related U.S. Application Data
(60) Provisional application No. 60/830,169, filed on Jul. 11, 2006.

(51) Int. Cl. A61F 2/06; A61M 25/10

(52) U.S. Cl. 623/1.11; 606/108; 606/28; 607/99

(57) ABSTRACT

A medical device such as a stent with a catheter, delivery balloon and a resistive heater for the balloon is disclosed. The resistive heater may be on top of, inside of the balloon or within the materials that make up the balloon. Resistive heaters may include metal foils, metallic printed circuits, and powdered conducting and semi-conducting metals.

### CROSS REFERENCE TO RELATED APPLICATION

This application claims the benefit of and incorporates by reference U.S. Patent Application No. 60/830,169 which was filed on Jul. 11, 2006.

### BACKGROUND OF THE INVENTION

1. Field of the Invention

This invention relates to implantable medical devices, such as stents.

2. Description of the State of the Art

This invention relates to radially expandable endoprostheses, which are adapted to be implanted in a bodily lumen. An “endoprosthesis” corresponds to an artificial device that is placed inside the body. A “lumen” refers to a cavity of a tubular organ such as a blood vessel.

A stent is an example of such an endoprosthesis. Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon, for example, by pumping a fluid into the catheter and balloon. To inflate or deflate the balloon, a physician typically uses an inflation device, such as a syringe, placed in fluid communication with the interior of the balloon. The physician uses one hand to grasp the syringe body and the other hand to maneuver the plunger to pressurize or depressurize the inflation fluid as required. Manually operated syringe-type inflation systems of the type described are manufactured and sold by Abbott Vascular of Santa Clara, Calif. under the trademark INDEFLATOR. After deployment, the balloon may then be deflated and the catheter withdrawn. In the case of a self-expanding stent, the stent may be secured to the catheter via a constraining member such as a retractable sheath or a sock. When the stent is in a desired bodily location, the sheath may be withdrawn which allows the stent to self-expand. The expansion of self-expandable stents may also be balloon-assisted. In this case, a balloon is inflated to partially expand the stent, but not enough to fully deploy the stent. The stent then fully expands and deploys without further assistance from a balloon.

Additionally, accurate stent placement is facilitated by real time visualization of the delivery of a stent. A cardiologist or interventional radiologist can track the delivery catheter through the patient's vasculature and precisely place the stent at the site of a lesion. This is typically accomplished by fluoroscopy or similar x-ray visualization procedures. For a stent, catheter, or balloon to be fluoroscopically visible they must be more absorptive of x-rays than the surrounding tissue. Alternatively, a fluid visible to magnetic resonance imaging (MRI) can be used. One way of accomplishing this is to use a fluid that is fluoroscopically visible to inflate a balloon. Such fluids are referred to as contrast agents.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil. In addition, the stent must possess sufficient flexibility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements of a pattern with respect to each other.

Additionally, a medicated stent may be fabricated by coating the surface of either a metallic or polymeric scaffolding with a polymeric carrier that includes an active or bioactive agent or drug. Polymeric scaffolding may also serve as a carrier of an active agent or drug.

Furthermore, it may be desirable for a stent to be biodegradable. In many treatment applications, the presence of a stent in a body may be necessary for a limited period of time until its intended function of, for example, maintaining vascular patency and/or drug delivery is accomplished. Therefore, stents fabricated from biodegradable, bioabsorbable, and/or bioerodable materials such as bioabsorbable polymers should be configured to completely erode only after the clinical need for them has ended.

A potential problem with polymeric stents is that their struts or bar arms can crack during crimping and expansion. This is especially the case with brittle polymers. The localized portions of the stent pattern subjected to substantial deformation during crimping and expansion tend to be the most vulnerable to failure.

Another potential problem with polymeric stents is creep. Creep is a consequence of the viscoelastic nature of polymeric materials. Creep refers to the gradual deformation that occurs in a polymeric material subjected to an applied load. Creep occurs even when the applied load is constant. Creep in a polymeric stent reduces the effectiveness of a stent in maintaining a desired vascular patency. In particular, creep contributes to recoil, allowing inward radial forces to permanently deform a stent radially inward.

Therefore, it is desirable for a stent to have flexibility and resistance to cracking during deployment. It is also advantageous for a stent to be rigid and resistant to creep after deployment.

### SUMMARY OF THE INVENTION

Apparatuses, methods and systems for delivering a stent at an elevated temperature are disclosed herein.

According to some embodiments, a method of delivering a stent mounted on an expandable member within a bodily lumen comprises: conveying energy to an expandable member coupled to a catheter assembly, heating the expandable member, allowing the heated expandable member to increase a temperature of a stent mounted on the expandable member, wherein the increase in temperature increases the flexibility of the stent such that formation of cracks in the stent upon its expansion is reduced or eliminated.

### BRIEF DESCRIPTION OF THE DRAWINGS

FIG. 1 depicts a stent.

FIG. 2A-2C depict a heated stent delivery system according to some embodiments of the invention.

FIG. 3A-3C depict a heated stent assembly according to some embodiments of the invention.

FIGS. 4A-4B depict a pressure delivery system according to some embodiments of the invention.

### DETAILED DESCRIPTION OF THE INVENTION

The structural members of polymeric stents can crack during crimping and radial expansion, sometimes leading to mechanical failure of a stent after deployment. Such cracking or rupturing can cause a stent strut to dislodge. The dislodged stent can cause an embolism in the lumen of the tubular organ. In addition, a dislodged stent can orient itself perpendicular to blood flow thereby causing thrombosis.

Rigid polymers are particularly susceptible to cracking when deformed such as when a stent is radially expanded. Polymers below their glass transition temperature tend to be rigid. The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semicrystalline polymer is exposed to an increasing temperature, the coefficient of expansion and the heat capacity of the polymer both increase as the temperature is raised, indicating increased molecular motion. As the temperature is raised the actual molecular volume in the sample remains constant, and so a higher coefficient of expansion points to an increase in free volume associated with the system and therefore increased freedom for the molecules to move. The increasing heat capacity corresponds to an increase in heat dissipation through movement. Tg of a given polymer can be dependent on the heating rate and can be influenced by the thermal history of the polymer. Furthermore, the chemical structure of the polymer heavily influences the glass transition by affecting mobility.

Below the Tg of a polymer, polymer segments may not have sufficient energy to move past one another. A polymer in a rigid state may be better suited to resist radial compressive forces in a stent once the stent is deployed. Thus, it would be advantageous, therefore, for the polymer of a stent to have a Tg that is above body temperature. However, such a polymer when it is below its Tg is susceptible to embrittlement and cracking during radial expansion.

As the temperature of a polymer is increased close to or above Tg, the energy barriers to rotation decrease and segmental mobility of polymer chains tends to increase. Consequently, polymers become more flexible, and thus, more resistant to embrittlement and cracking when they are at a temperature that is close to or above Tg. Therefore, it may be more desirable for a polymeric stent to be close to or above the Tg of the polymer when a stent is expanded.

However, polymers tend to be more susceptible to creep when they are close to or above the Tg. Additionally, creep can also result in a polymeric stent under stress. In particular, creep allows inward radial forces to permanently deform a stent radially inward. Therefore, creep reduces the effectiveness of a stent in maintaining a desired vascular patency.

In general, it is desirable to have a delivery system that allows a stent to be at least: (1) flexible and resistant to cracking during expansion; and (2) rigid and resistant to compressive forces so as to maintain vascular patency after deployment at an implant site.

Various embodiments of a method and system for delivering a stent in a bodily lumen that meet these criteria are disclosed. In general, the criteria may be met in part by using a stent fabricated from a polymer that is rigid at body temperature, e.g., a polymer with a Tg greater than body temperature. The other criterion may be met by increasing the flexibility of the stent during expansion such that formation of cracks in the stent upon its expansion is reduced or eliminated. This may be accomplished by heating a stent to a temperature close to, at, or above its Tg before, during and/or after deployment.

The invention provides for heating a stent upon deployment to dramatically improve the mechanical stability of a deployed polymeric stent. By inflating the stent at an elevated temperature, the deployed stent exhibits less cracking, better mechanical properties, and a more uniformity to the stent structure. Traditional techniques for deploying a stent at an elevated temperature require the use of heated contrast media, which is cumbersome and difficult in practice, so the technique has failed to gain acceptance as a potential technique.

The invention reduces cracks in polymeric stents during deployment by providing a balloon catheter that contains a heating mechanism, which enables the heating of an attached polymeric stent before, during and/or after deployment of the stent into the lumen. Specifically, the invention provides a method for resistively heating a delivery balloon that combines ease of use and a reproducible mechanism.

According to Ohm's Law, electric current passing through a conductor is directly proportional to the potential difference across it, when the temperature is constant. This constant of proportionality is the resistance of the conductor. For many electric conductors, the current flowing through them is directly proportional to the applied voltage. Ohm's Law depends upon the fact that the velocity of charges through the material is proportional to the electric field in the conductor. The ratio of voltage to current is called the resistance, and if the ratio is constant over a wide range of voltages, the material is said to be an “ohmic” material. If the material can be characterized by such a resistance, then the current can be predicted from: Volts=Amperes×Ohms.

A resistor is a part of an electrical circuit and is used to control the flow of electrical current and has specific resistance, which is measured in ohms. A heater consists of one or more electrically conductive foils connected to an electrical supply. The length of the circuit and the temperature determine the resistance of the conductor. Series heaters, are highly efficient resistive heating circuits where the heating effect is generated within a resistive metal core.

In certain embodiments, a delivery balloon can include a resistive heater such as a resistive heating element or material for heating the balloon. The resistive heating element or material can be in electrical communication with a power source. The electrical power from the power source can be used to increase the temperature of the heating element or material, thereby heating the balloon material.

In some embodiments, the resistive heater can be a layer of electrically conductive material on at least a portion of an inside or outside surface of a balloon. In an embodiment, the conductive layer is a metal or metallic layer and includes aluminum, copper, or gold, but is not limited thereto. Other materials, such as a conductive adhesive, may also be included in the conductive layer.

In other embodiments, the resistive heater can be a conductive material dispersed within the balloon material. The conductive material can be, for example, carbon fiber, or powdered conducting or semi-conducting metals, polymers, or graphite.

In further embodiments, the resistive heater can be a conductive filament or wire wrapped or coiled on the outside, inside, or within the balloon material. In some embodiments, polymeric materials are formed around resistance heating wire by injection molding techniques. In several embodiments, the resistive heater includes a resistance heating material, such as a resistance heating wire, or, a resistance heating material, such as a foil or printed circuit. In one embodiment, a laser trimmed or photoetched Al or Cu foil could be employed. In some embodiments, the resistance heating wire includes a Ni—Cr alloy, although copper, steel, and stainless-steel alloy wires could be suitable. In several embodiments, the resistance heating wire is terminated with flat electrical conductors for connection to a power source.

In additional embodiments, the balloon material can be fabricated from at least in part of a conductive polymer, or polymer blend, such as a conductive nylon.

The material for use in fabricating the balloon may be any material capable of absorbing resistive heat generated by a resistive heater with or on the balloon. Balloon materials are typically elastermeric polymers. An exemplary commercial embodiment includes Pebax®. The heating layer may be a conductive thin film applied to the surface of an insulative substrate.

The heated balloon material should be able to heat an attached stent prior to, during, or after stent deployment. In several embodiments, the balloon surface is fabricated to facilitate the localization of the heating effect on an attached stent. This may be accomplished by selectively coating portions the balloon's inner surface with a metallic layer so that only portions of the balloon are heated.

In one embodiment, the stent is heated before, during, or after deployment with a balloon catheter into the bodily lumen. The stent can optionally be heated after expansion by keeping the balloon inflated while continuing the resistive heating, thereby continuing to heat the stent.

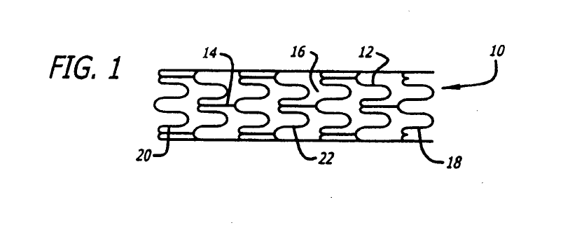

Referring to FIG. 1, a stent 10 is illustrated. Stent 10 can include a plurality of struts 12 connected by linking struts 14, with interstitial spaces 16 located in between the struts. The plurality of struts 12 can be configured in an annular fashion in discrete “rows” such that they form a series of “rings” throughout the body of stent 10. Thus, stent 10 can include a proximal ring 18, a distal ring 20 and at least one central ring 22.

As indicated above, stent 10 may be made from and/or coated with a biostable polymer or a bioerodable, biodegradable, bioadsorbable polymer or any combination thereof. In some embodiments, a polymeric stent can include other materials, such as layers or deposits of metallic material which can be bioerodable. The pattern should not be limited to what has been illustrated as other stent patterns are easily applicable with embodiments of the present invention.

In general, a stent pattern is designed so that the stent can be radially compressed (crimped) and radially expanded (to allow deployment). The stresses involved during compression and expansion are generally distributed throughout various structural elements of the stent pattern. As a stent expands, various portions of the stent can deform to accomplish a radial expansion.

Additionally, fabrication of an implantable medical device, such as a stent, may include forming a pattern that includes a plurality of interconnecting structural elements or struts on a tube. Polymer tubes may be formed by various types of methods, including, but not limited to extrusion or injection molding. In some embodiments, the diameter of the polymer tube prior to fabrication of an implantable medical device may be between about 0.2 mm and about 5.0 mm, or more narrowly between about 1 mm and about 3 mm. In some embodiments, forming a pattern on a tube may include laser cutting a pattern on the tube. Representative examples of lasers that may be used include, but are not limited to, excimer, carbon dioxide, and YAG. In other embodiments, chemical etching may be used to form a pattern on a tube.

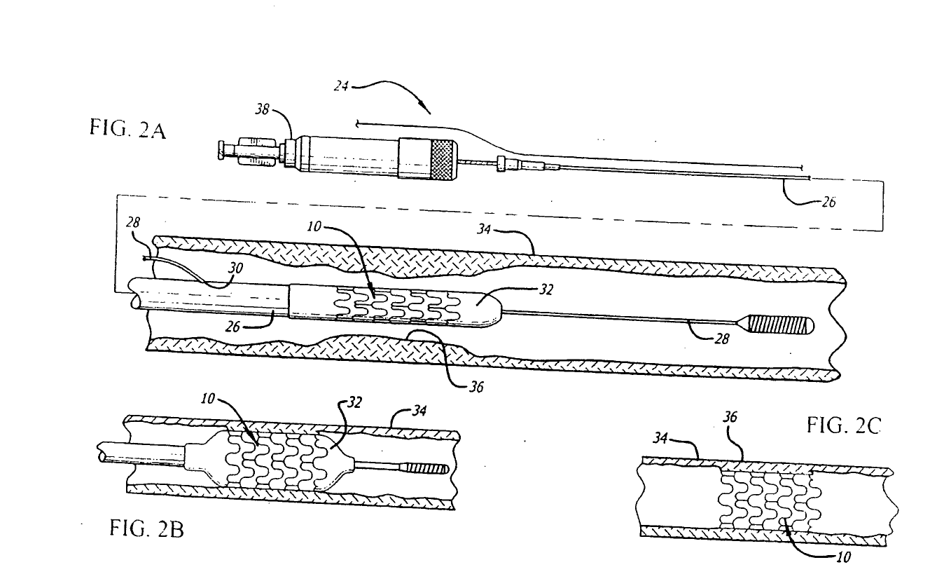

To deliver a stent to a vessel, the stent is mounted on a balloon-catheter assembly. FIGS. 2A-2C illustrate an exemplary delivery system for delivering a balloon expandable stent into a tubular organ 34 according to several embodiments of the invention. FIG. 2A illustrates a catheter 24 which includes a proximal end 38 and a tip 26 on which an expandable member 32, e.g., a balloon, can be mounted on expandable member 32, the inside of which is coated with a metallic layer (not shown). An opening 30 is located on tip 26 through which a guidewire 28 is inserted to aid the user in the delivery of stent 10 which is mounted on expandable member 32.

In one deployment application, tip 26 of catheter 24, with a stent-balloon assembly mounted thereon, can be inserted into tubular organ 34 to treat a targeted region 36 of tubular organ 34. Once the stent-balloon assembly is positioned at targeted region 36, the expandable member 32 is heated resistively using a resistive heater in or on expandable member 32 (not shown) which is connected to catheter 24, thereby heating stent 10. The stent-balloon assembly can be expanded by conveying a fluid into expandable member 32 from a fluid source (not shown in this figure) (see FIG. 2B). The fluid source can be an inflation device capable of conveying fluid into the catheter by creating a pressure gradient between the inflation device and the catheter. Subsequent to the positioning and expansion of stent 10, expandable member 32 can be deflated and withdrawn from targeted region 36 (see FIG. 2C) leaving stent 10 at targeted region 36.

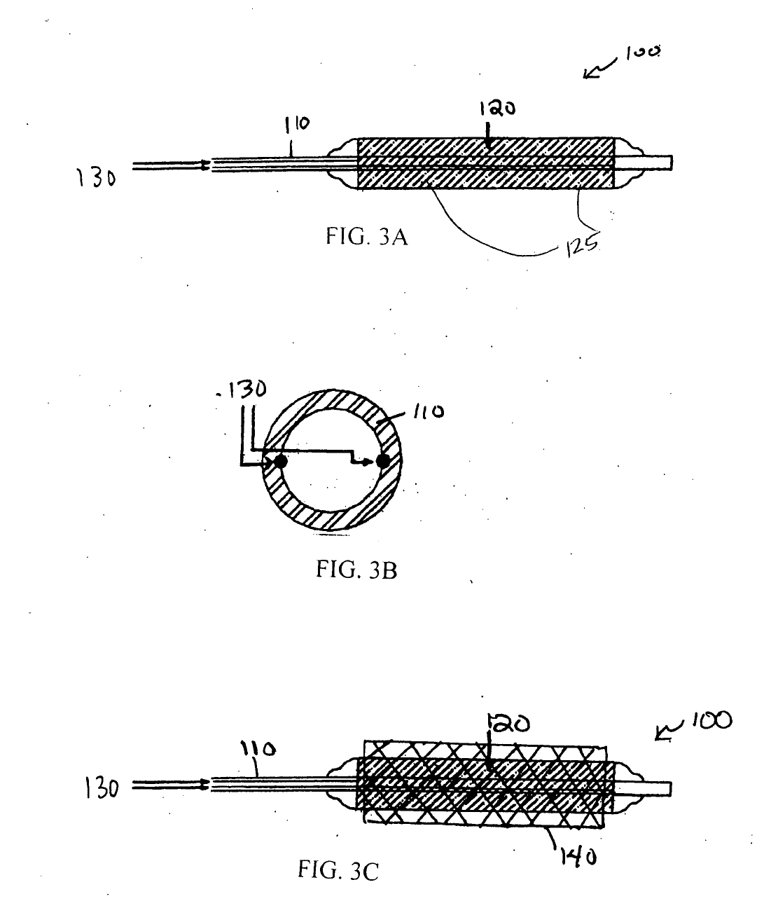

Electrical resistance heating is an in-situ electrical heating technology that uses electricity to heat a delivery balloon. FIGS. 3A-3C depict a heated balloon assembly according to several embodiments of the invention. FIG. 3A depicts a side view of a balloon-catheter assembly 100. Balloon-catheter assembly 100 includes a catheter 110 and a support element or a delivery balloon 120 attached to catheter 110. In one embodiment, delivery balloon 120 is coated with a very thin metal layer 125 useful for facilitating the heating of balloon 120 before, during, or after stent deployment. Power may be supplied to metal coating 125 of balloon 120 through one or more wires 130 to resistively heat metal layer 125 of balloon 140. In one embodiment, metal layer 125 on balloon 120 may be connected to a power supply by wires located in the tubing of catheter 110. The resistive heating of balloon 120 results in the heating of attached polymeric stent 120, which aids in stent deployment, thereby reducing cracking in the stent caused by deployment. Wires 130 for the balloon heating element may be integrated with balloon 120, which automates stent heating.

FIG. 3B depicts a cross sectional view of catheter 110 that includes wires 130 for use in carrying power to the balloon-catheter assembly 100 to heat balloon 120 according to one embodiment of the invention. The wires may be co-extruded with the catheter 110, such that the wires are at least partially embedded in the construct of catheter 110. In one embodiment, the wires 130 are metallic. Wires 130 can extend within catheter 110 to a position between balloon 120 and the proximal end of catheter 110. Proximal ends of wires 130 can be connected to a power source (not shown) which supplies electrical power to metal layer 125 through wires 130.

FIG. 3C depicts a side view of balloon-catheter assembly 100 including wires 130 embedded in catheter 110 and a stent 140 crimped onto the balloon-catheter assembly 100 according to one embodiment of the invention.

Electrical power is transmitted through wires 130 to metallic layer 120 which is resistively heated, thereby heating balloon 120. Heated balloon 120 then heats stent 140. In one embodiment, the stent can be heated prior to expanding the stent. Alternatively, the heating can be started during expansion of the stent. In another embodiment, the stent can be heated prior to expansion and heating can continue during and optionally after expansion. In such embodiments, the stent can be heated to the target temperature prior to expansion. Alternatively, the stent can be heated to a temperature below the target temperature prior to expansion and heated to the target temperature during or after expansion.

In some embodiments, the stent can be heated to a target temperature at a relatively constant rate. Alternatively, the heating rate of the stent can be nonlinear, e.g., slow initially and fast subsequently. In a further embodiment, the heating can be performed in states or step-wise with equilibrium periods between increases in temperature.

Additionally, the temperature of the balloon or stent can be monitored during the heating process. In one embodiment, thermocouple leads (not shown) can be coupled within or on the balloon to monitor the temperature. The leads can be in communication through wires extending with the catheter with a system that displays the temperature.

In some embodiments, the heating can be manually controlled by an operator. In other embodiments, the heating can be preprogrammed into a control system that automatically adjusts the heating rate. In some embodiments, the heating rate and/or temperature of the stent can be controlled by a control system. The power source and temperature sensors can be in communication with the control system. Based on input parameters, such as measured temperature, the heating can be controlled to obtain desired heating of the stent. The control system can employ a feedback control mechanism known to one of ordinary skill in the art.

The stent is sufficiently heated for a specified time to heat the stent to a target temperature that depends on the polymer in the stent. In one embodiment, the resistive heater heats the stent to a temperature between body temperature, 37° C., and the Tg of the stent polymer. In this embodiment, fewer cracks in the stent result from deployment because the stent is sufficiently heated above body temperature to reduce cracking. In another embodiment, the temperature of the stent is heated to a temperature above the glass transition temperature of the stent. The resistive heater in or on the balloon may be controlled to adjust the temperature to a desired or an optimum temperature for stent deployment. In one embodiment, the optimum temperature is the temperature at which the least number of cracks are produced. After the stent is deployed, balloon 120 is deflated and removed, leaving behind a radially expanded stent that exhibits fewer cracks than a stent that has not been heated prior to or during deployment.

The present invention can be generally applied to, but is not limited to, self-expandable stents, balloon-expandable stents, stent-grafts, vascular grafts, and generally tubular implantable medical devices. A stent specifically designed and intended solely for localized delivery of a therapeutic agent is also within the scope of this invention. Thus, the stent may also be used to open a lumen within an organ in a mammal, maintain lumen patency, and/or reduce the likelihood of the narrowing of a lumen.

In one embodiment, the polymer for use in forming the stent scaffolding and/or the stent coating may be configured to degrade after implantation by fabricating the stent either partially or completely from biodegradable polymers. A biodegradable stent may remain in the body until its intended function of, for example, maintaining vascular patency and/or drug delivery is accomplished. For biodegradable polymers used in coating applications, after completion of the process of degradation, erosion, and absorption, no polymer remains on the stent. In some embodiments, very negligible traces of residue are left behind. In one embodiment, poly(L-lactide) is used to fabricate the stent.

It is understood that after the process of degradation, erosion, absorption, and/or resorption has been completed, no part of the stent will remain or in the case of coating applications on a biostable scaffolding, no polymer will remain on the device. In some embodiments, very negligible traces or residue may be left behind. For stents made from a biodegradable polymer, the stent is intended to remain in the body for a duration of time until its intended function of, for example, maintaining vascular patency and/or drug delivery is accomplished.

Representative examples of polymers that may be used to fabricate or coat an implantable medical device include, but are not limited to, poly(N-acetylglucosamine) (Chitin), Chitosan, poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), polyester amide, poly(glycolic acid-co-trimethylene carbonate), co-poly(ether-esters) (e.g. PEO/PLA), polyphosphazenes, biomolecules (such as fibrin, fibrinogen, cellulose, starch, collagen and hyaluronic acid), polyurethanes, silicones, polyesters, polyolefins, polyisobutylene and ethylene-alphaolefin copolymers, acrylic polymers and copolymers other than polyacrylates, vinyl halide polymers and copolymers (such as polyvinyl chloride), polyvinyl ethers (such as polyvinyl methyl ether), polyvinylidene halides (such as polyvinylidene chloride), polyacrylonitrile, polyvinyl ketones, polyvinyl aromatics (such as polystyrene), polyvinyl esters (such as polyvinyl acetate), acrylonitrile-styrene copolymers, ABS resins, polyamides (such as Nylon 66 and polycaprolactam), polycarbonates, polyoxymethylenes, polyimides, polyethers, polyurethanes, rayon, rayon-triacetate, cellulose, cellulose acetate, cellulose butyrate, cellulose acetate butyrate, cellophane, cellulose nitrate, cellulose propionate, cellulose ethers, and carboxymethyl cellulose. Another type of polymer based on poly(lactic acid) that can be used includes graft copolymers, and block copolymers, Such as AB block-copolymers (“diblock-copolymers”) or ABA block-copolymers (“triblock-copolymers”), or mixtures thereof.

Additional representative examples of polymers that may be especially well suited for use in fabricating or coating an implantable medical device include ethylene vinyl alcohol copolymer (commonly known by the generic name EVOH or by the trade name EVAL), poly(butyl methacrylate), poly(vinylidene fluoride-co-hexafluoropropylene) (e.g., SOLEF 21508, available from Solvay Solexis PVDF, Thorofare, N.J.), polyvinylidene fluoride (otherwise known as KYNAR, available from ATOFINA Chemicals, Philadelphia, Pa.), ethylene-vinyl acetate copolymers, and polyethylene glycol.

Implantable medical devices such as stents are typically subjected to stress during use, both before and during treatment. “Use” includes, but is not limited to, manufacturing, assembling (e.g., crimping a stent on a catheter), delivery of a stent into and through a bodily lumen to a treatment site, and deployment of a stent at a treatment site. Both a scaffolding and a coating on a scaffolding experience stress that result in strain in the scaffolding and/or coating. For example, during deployment, the scaffolding of a stent can be exposed to stress caused by the radial expansion of the stent body. In addition, the scaffolding and/or coating may be exposed to stress when it is mounted on a catheter from crimping or compression of the stent.

In some embodiments, the bending portions of the stent are selectively heated because they are subjected to the most stress. In several embodiments this may be accomplished by selectively coating the balloon with metal such that only select portions of the balloon and an attached stent are heated.

Implantable medical devices, such as stents, that relate to the embodiments described herein typically include an underlying scaffolding or substrate. The underlying structure or substrate of the device can be of virtually any design. The substrate may have a polymer-based coating that may contain, for example, an active agent or drug for local administration at a diseased site. The active agent can be any substance capable of exerting a therapeutic or prophylactic effect.

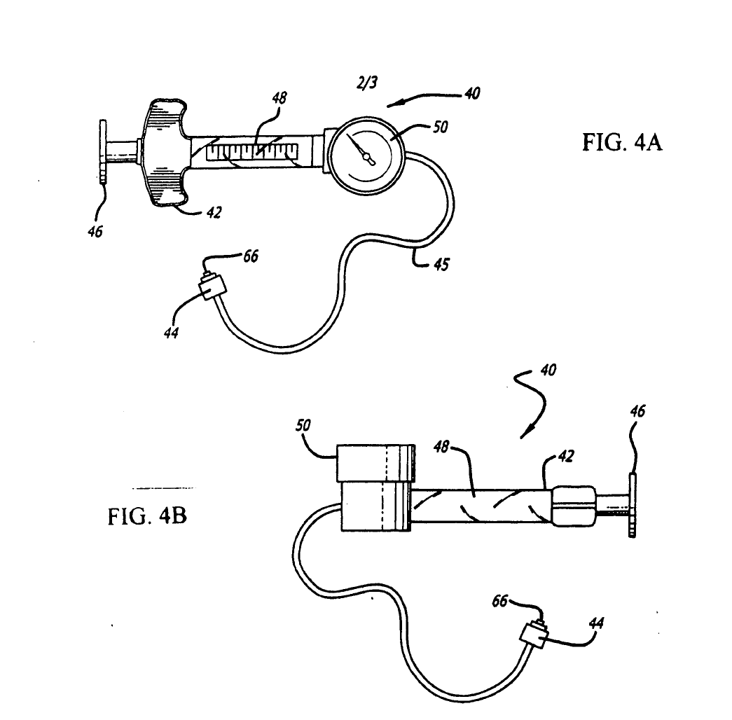

FIGS. 4A-4B illustrate an exemplary inflation device 40 which can be used to expand a stent-balloon assembly. Inflation device 40 can convey fluid into catheter 24 and expand expandable member 32 by creating a pressure gradient between inflation device 40 and catheter 24. Inflation device 40 can include a pressure injector at a first end 42 and an exit port 66 at the tip of a flexible tube 45. Inflation device 40 further includes a chamber 48 and a pressure gauge 50. Chamber 48 can be filled with a fluid for use in expanding an expandable member 32. Typically, the fluid is a liquid, however, a gas may also be used. Chamber 48 can have a capacity, for example, between about 10 ml and 40 ml.

In addition to expanding a balloon, the fluid may assist a user in visualizing the catheter and balloon during delivery. As indicated above, a fluid that is visible to an imaging technique, such as x-ray fluoroscopy or magnetic resonance imaging (MRI), may be used to inflate a balloon. Such fluids are referred to as contrast agents. Thus, a contrast agent can include a radiopaque agent or a magnetic resonance imaging agent. “Radiopaque” refers to the ability of a substance to absorb x-rays. An MRI agent has a magnetic susceptibility that allows it to be visible with MRI.

In some applications, exit port 66 of inflation device 40 can be coupled to a catheter for inflation (and deflation) of a balloon during deployment of a stent. A fluid for inflating expandable member 32 can be disposed in chamber 48. During a delivery procedure, the pressure injector 46 can be depressed for injecting fluid 68 into catheter 24 and expandable member 32 for inflation thereof. Pressure gauge 50 gives a user an indication of the pressure so that it can be monitored to prevent balloon burst. Inflation device 40 can be made of a durable plastic such as polycarbonate or metal such as stainless steel. Moreover, inflation device 40 may be reusable or disposable. Device 40 is typically disposable after a one single use due to the highly critical nature of the catherization procedure. Generally, inflation device 40 can be between four inches to 10 inches in length.

In some embodiments, balloon expansion may be performed in stages or measured intervals. Such a procedure tends to reduce or prevent vessel injury. Additionally, performing the expansion in stages may allow a stent to equilibrate at higher temperatures as it is heated.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A delivery assembly comprising:
a catheter; and
an expandable member attached to the catheter, the expandable member comprising a resistive heater for resistively heating the expandable member, wherein the heat generated is configured to heat a stent when the stent is mounted on the expandable member, thereby heating the stent to a temperature above body temperature.

2. The delivery assembly of claim 1, wherein the expandable member is a delivery balloon.

3. The delivery assembly of claim 1, wherein the heat increases the temperature of the stent close to, at, or above a glass transition temperature of the polymer of the stent.

4. The delivery assembly of claim 1, wherein the expandable member is adapted to receive a fluid for expanding the expandable member.

5. The delivery assembly of claim 1, wherein the resistive heater comprises a metallic compound is dispersed in a material making up the expandable member.

6. The delivery assembly of claim 1, wherein the resistive heater comprises a metal or metallic layer disposed on the expandable member.

7. The delivery assembly of claim 1, wherein the resistive heater is in electrical communication with a power source.

8. The delivery assembly of claim 1, further comprising wires embedded within the catheter, wherein the wires are connected to the resistive heater and are adapted to provide power from a power supply to the resistive heater.

9. The delivery assembly of claim 6, wherein the metal or metallic layer is selectively disposed on the expandable member to allow selective heating of selective portions of the stent.

10. The delivery assembly of claim 1, wherein the resistive heater comprises materials selected from the group consisting of metal foils, metallic printed circuits, powdered conducting metals, powdered semi-conducting metals, and combinations thereof.

11. The delivery assembly of claim 1, wherein the resistive heater comprises metallic wire embedded in the expandable member.

12. The delivery assembly of claim 1, further comprising a power supply connected to the catheter and adapted to resistively heat the expandable member.

13. The delivery assembly of claim 1, further comprising a polymeric stent disposed over the expandable member.

14. The delivery assembly of claim 13, wherein the stent comprises a biostable and/or bioabsorbable polymer.

15. A method of delivering a stent mounted on an expandable member within a bodily lumen, comprising:
heating an expandable member attached to a catheter, the expandable member comprising a resistive heater for resistively heating the expandable member;
allowing the heated expandable member to increase the temperature of a polymeric stent disposed on the expandable member; and
expanding the expandable member, wherein the increase in temperature increases the flexibility of the stent such that formation of cracks in the stent upon its expansion is reduced or eliminated.

16. The method of claim 15, wherein the expandable member is expanded by fluid from a fluid source that comprises an inflation device capable of conveying fluid into the expandable member by creating a pressure gradient between the inflation device and the catheter.

17. The method of claim 15, wherein the resistive heater comprises a metal or metallic layer disposed on the expandable member.

18. The method of claim 15, further comprising wires embedded within the catheter, wherein the wires are connected to the resistive heater and are adapted to provide power from a power supply to the resistive heater.

19. The method of claim 15, further comprising a power supply connected to the catheter and adapted to provide power to the resistive heater to resistively heat the expandable member.

20. The method of claim 15, wherein the expandable member comprises a catheter balloon.

21. The method of claim 15, further comprising positioning the disposed stent at an implant site.

22. The method of claim 21, wherein the stent comprises a biostable and/or bioabsorbable polymer.

23. The method of claim 15, wherein the resistive heater heats the expandable member to a temperature that allows the expandable member to increase the temperature of the stent to a temperature close to, at, or above a glass transition temperature of the polymer of the stent.

24. A system for delivering a stent mounted on an expandable member within a bodily lumen, comprising:
a catheter;
an expandable member attached to the catheter, the expandable member comprising a resistive heater for resistively heating the expandable member, wherein the expandable member is capable of absorbing heat energy and transferring heat;
a polymeric stent attached to the expandable member; and
a fluid source for providing fluid to inflate the expandable member, wherein the resistive heating of the expandable member is adapted to increase the temperature of the stent thereby increasing the flexibility of the attached stent such that formation of cracks in the stent upon its expansion is reduced or eliminated.

25. The system of claim 24, wherein the fluid source comprises an inflation device capable of conveying fluid into the catheter by creating a pressure gradient between the inflation device and the catheter.

26. The system of claim 24, wherein the heat from the expandable member increases the temperature of the stent close to, at, or above a glass transition temperature of the polymer of the stent.

27. The system of claim 24, wherein the stent comprises a biostable and/or bioabsorbable polymer.

28. The system of claim 24, further comprising a power supply in electrical communication with the resistive heater to resistively heat the expandable member.

29. The system of claim 28, wherein catheter comprises connectors from transmit power for the power supply to the resistive heater.
